(19) United States
(12) Patent Application Publication
(10) Pub. No.: US 2023/0131129 A1
(43) Pub. Date: Apr. 27, 2023

(54) STENT

(71) Applicant: optimed medizinische Instrumente GmbH, Ettlingen (DE)

(72) Inventors: Thilo WACK, Tholey (DE); Thomas WILLE, Senftenberg (DE); Fabian SCHMIDT, Karlsruhe (DE); Achim ZIPSE, Baden-Baden (DE)

(21) Appl. No.: 17/970,765

(22) Filed: Oct. 21, 2022

(30) Foreign Application Priority Data
Oct. 22, 2021 (DE) 10 2021 127 509.1

(51) Int. Cl. A61L 27/58; A61F 2/90; A61F 2/07; A61L 27/04

(52) U.S. Cl. CPC A61L 27/58; A61F 2/90; A61F 2/07; A61L 27/047; A61F 2210/0004; A61F 2230/0017

(57) ABSTRACT

The invention relates to a stent for transluminal implantation into hollow organs, in particular into blood vessels, ureters, esophagi, the colon, the duodenum, the airways or the biliary tract, comprising an at least substantially tubular body that extends along a longitudinal direction and that can be converted from a compressed state having a first cross-sectional diameter into an expanded state having an enlarged second cross-sectional diameter. The stent in accordance with the invention is characterized in that the tubular body comprises an inner body and an outer body, with the outer body surrounding the inner body at least regionally, with the outer body completely running around at least one section of the inner body, and the outer body is formed from a bioresorbable material or comprises a bioresorbable material.

The present invention relates to a stent for transluminal implantation into hollow organs, in particular into blood vessels, ureters, esophagi, the colon, the duodenum, the airways or the biliary tract, comprising an at least substantially tubular body that extends along a longitudinal direction. The tubular body can be converted from a compressed state having a first cross-sectional diameter into an expanded state having an enlarged second cross-sectional diameter that is enlarged in comparison with the first cross-sectional diameter.

Stents are used to treat pathologically altered hollow organs, for example, when the hollow organs have narrowed (stenosis). Another field of application is the treatment of aneurysms. For the treatment, the stent is introduced in the compressed state via an insertion catheter to the position within the hollow organ to be treated where the stent is expanded by dilatation or by self-expansion to a diameter that e.g. corresponds to the diameter of the healthy hollow organ so that, for example, a supporting effect of the hollow organ, in particular of a vessel wall, is achieved.

It is in particular desirable for stents that the stents provide a high radial deployment force, i.e. have a sufficiently large supporting effect. At the same time, it is desired that the stents are flexible along the longitudinal direction to follow movements of the hollow organ.

In practice, however, a compromise often has to be found between the radial deployment force and the flexibility of the stent.

Furthermore, it is important that the stent can be easily inserted into a hollow organ and can be placed there as accurately as possible.

It is therefore the underlying object of the invention to provide an improved stent that in particular takes into account the aforementioned aspects.

This object is satisfied by a stent in accordance with claim 1.

The stent in accordance with the invention is characterized in that the tubular body comprises an inner body, in particular a tubular inner body, and an outer body, in particular likewise a tubular body, with the outer body surrounding the inner body at least regionally, with the outer body completely running around at least one section of the inner body, and the outer body is formed from a bioresorbable material or comprises a bioresorbable material.

In this respect, the invention is based on the recognition that, due to the outer body composed of a bioresorbable material, advantages can be achieved during the insertion of the stent and, likewise after the degradation of the bioresorbable material, there are further advantages when the inner body permanently remains in the hollow organ.

After the insertion of the stent into the hollow organ, the outer body composed of the bioresorbable material is degraded, i.e. “resorbed,” over a certain period of time. After the time period has elapsed, only the inner body therefore in particular remains in the hollow organ. On the insertion, the advantage can thus result that the outer body simplifies the insertion in that the outer body can, for example, be shaped such that it can be easily fastened to an insertion set of instruments. Likewise, it is, for example, possible that a more precise positioning of the inner body and thus of the stent becomes possible due to the outer body, as described in more detail later.

In accordance with the invention, the outer body surrounds the inner body at least regionally. This means that the outer body e.g. completely runs around the longitudinal direction in at least some regions of the stent. This in particular applies to the expanded state and the compressed state. The outer body can, for example, hold the inner body in the compressed state. The outer body can, for example, be formed by a wire placed around the stent sections or by a wire mesh or can comprise such a wire or a wire mesh. Instead of the wire, a thread can also be used that is supported by the inner body.

The inner body, unlike the outer body, can be formed from a material permanently durable in the hollow organ, for example, from nickel-titanium alloys (nitinol), from cobalt-chromium alloys, cobalt-nickel alloys, or platinum-chromium alloys. The inner body can in particular be formed from a shape-storing material (“shape memory”) that assumes the stored shape from a limit temperature onward.

For a simpler positioning under X-ray observation, the stent can comprise a plurality of X-ray markers, in particular composed of tantalum. The X-ray markers can, for example, be fastened to the ends of the inner body and/or of the outer body, viewed in the longitudinal direction. X-ray markers at the outer body can in particular be formed by thickened material portions of the bioresorbable material.

At least one X-ray marker, in particular in the form of an eyelet, preferably extends away from at least one end of the inner body in the longitudinal direction, wherein the X-ray marker has an asymmetrical shape. A marker can be a section of the inner body that has an increased radiopacity, i.e. is particularly easily visible in an X-ray. The marker can in particular be an eyelet that is, for example, filled with or covered by the aforesaid tantalum.

The stent is tubular and accordingly, in the expanded state, can have an empty, continuous volume within the inner body through which e.g. a blood flow is possible. The stent can, over its total length in the longitudinal direction, have a cross-sectional diameter that is at least substantially constant in the expanded state. In cross-section, the stent can have a circular or elliptical shape.

Furthermore, the length of the stent in the longitudinal direction can be greater (e.g. at least 2×, 5×, 10×, or 30× greater) than the cross-sectional diameter.

The inner body preferably has a smaller cross-sectional diameter than the outer body in both the expanded state and the compressed state.

Instead of an inner body and an outer body, an inner structure and an outer structure could also be spoken of. In each case, the term “body” is intended to illustrate that these structures (even if they are e.g. divided into different stent sections) belong together and can together e.g. bring about the support of the hollow organ.

Advantageous further developments of the invention can be seen from the description, from the drawings and from the dependent claims.

In accordance with a first embodiment, the inner body comprises a plurality of annular sections that are in particular separate from one another. The separate annular sections can accordingly not be connected to one another by material of the inner body. After the degradation of the bioresorbable material, in particular only the annular sections thus remain in the hollow organ. If the annular sections are not connected to one another, the inner body then remaining in the hollow organ allows a maximum flexibility and adaptability to movements and deformations of the hollow organ, wherein a good supporting effect can, however, simultaneously be achieved by the individual rings. Without the at least temporarily present bioresorbable outer body, it is almost impossible to place each of the individual annular sections individually and at a constant spacing from one another in the hollow organ, which is difficult in practice, in particular for self-expanding structures. Due to the individual annular sections, the high flexibility of the remaining inner body then results after the resorption of the outer body.

Due to the stent described herein, it is now permitted to simultaneously insert many separate annular sections into a hollow organ, whereby an advantage results during the insertion. Nevertheless, after the degradation of the bioresorbable material, a great supporting effect and flexibility are achieved by the parts of the inner body that remain permanently in the body.

In accordance with a further embodiment, the annular sections are held by the outer body. As already indicated above, the outer body can therefore, for example, keep the annular sections at a spacing, in particular an equal spacing, from one another along the longitudinal direction. Due to the equal spacing of the annular sections from one another, a uniform supporting effect results in the hollow organ. Due to the holding of the annular sections by means of the outer body, the annular sections are furthermore also prevented from changing their relative positions during the insertion.

At least one of the annular sections preferably comprises a row of cells that follow one another in the peripheral direction of the stent and that preferably form a closed ring running around in the peripheral direction of the stent. The ring preferably has a cross-section, which is closed in annular form and which is formed by the cells of the ring, perpendicular to the longitudinal direction of the stent. An ideal radial deployment force and supporting effect of the stent or of the inner body can be provided in the support section/stent section by such a ring that runs around in the peripheral direction and that is preferably closed. An annular section can in principle be formed by any desired cells. An annular section can in particular have exactly one single row or exactly two or three rows of cells connected to one another. Due to two or three rows of cells connected to one another, the annular section is extended in the longitudinal direction, but also provides an increased deployment force.

In accordance with a further embodiment, the inner body and/or the outer body is/are each an independent stent. The inner body and/or the outer body could therefore be considered as independent stents. This means that each of these bodies could be inserted alone into a hollow organ and would bring about a supporting effect in the hollow organ if the respective body has been transferred from the compressed state having a first cross-sectional diameter into the expanded state having an enlarged second cross-sectional diameter. Here, it is advantageous that the known manufacturing processes for stents can be separately applied to the inner body and/or the outer body. After the manufacture of the inner body and the outer body, the inner body can then be inserted into the outer body to form the complete stent.

In accordance with a further embodiment, the inner body is self-expanding and/or the outer body is balloon-expandable. If the inner body were considered alone, the inner body would independently unfold from the compressed state into the expanded state if there were no pressure from the outside. The outer body, in contrast, cannot be self-expanding and is instead balloon-expandable, for example. This means that a balloon can be placed within the outer body, wherein the balloon is then inflated under pressure, enlarges and, during this enlargement, urges and transfers the outer body from the compressed state into the expanded state (“balloon dilation”). The inner body and the outer body can in particular be applied to the balloon. If the inner body is self-expanding and the outer body is balloon-expandable, the inner body presses against the outer body from the inside in the compressed state of the entire stent and is fastened, at least in part, to the outer body in this manner, in particular by a force fit.

The force for transferring the outer body from the compressed state into the expanded state is in particular greater than the force with which the inner body presses against the outer body from the inside. Accordingly, the stent as a whole can be considered as balloon-expandable, wherein the inner body is in particular held by the outer body in the compressed state.

Alternatively, it is also possible for both the inner body and the outer body to be self-expanding or balloon-expandable.

In accordance with a further embodiment, the inner body comprises a plurality of cells that are defined by bordering elements formed by the inner body. Alternatively or additionally, the outer body can comprise a plurality of cells that are defined by bordering elements formed by the outer body.

The bordering elements (e.g. so-called “struts”) can be produced by removing material when cutting the inner body and/or the outer body from a material, in particular a tubular material, wherein the bordering elements or struts remain. The bordering elements are preferably fixedly connected to one another and in particular in one piece. The inner body and/or the outer body accordingly e.g. does/do not comprise a braided material.

A cell can be connected to one or more other cells by one connection section of by a plurality of connection sections. A cell comprises said recess as well as its respective bordering elements, wherein the connection sections belong to the bordering elements.

In accordance with a further embodiment, cells of the inner body and/or of the outer body form a convex polygon and in particular have a diamond shape. The aforementioned shape of the cells can in particular result when the cells are placed or pressed onto a plane (the so-called unwinding). In a convex polygon, all the interior angles each have an angle of ≤180°. Such cells can also be called closed cells. Due to the shape of a convex polygon and in particular due to the diamond shape, a high deployment force and thus a high supporting effect of the stent can result.

Most or all of the cells in particular have the shape of a convex polygon or a diamond shape.

Alternatively or additionally, cells of the inner body and/or of the outer body can also form a concave polygon. In a concave polygon, at least one interior angle can have an angle of >180°. For example, at least some of the bordering elements of a respective cell can have a zigzag shape. Due to such zigzag cells or generally due to cells having the shape of a concave polygon, the flexibility of the stent can be further increased. Most or all of the cells can also have the shape of a concave polygon.

In accordance with a further embodiment, in particular the majority or all the cells of the inner body and the outer body have the same or a similar shape, wherein the cells of the inner body and the outer body at least substantially lie approximately congruently on one another or are arranged offset from one another. A similar shape can in particular be spoken of when the bordering elements of two cells have a maximum offset when they lie on one another that does not exceed 10%, 20% or 30% of the length of the greatest extent of a cell in one spatial direction.

If the cells of the inner body and the outer body are arranged offset from one another, connection sections of the cells of the outer body can each be arranged centrally between two connection sections of the cells of the inner body. In this way, a “phase shift” occurs between the cells of the inner body and the outer body. Due to the cells arranged offset from one another, the advantage of a higher wall coverage of the hollow organ results. This means that the regions of the hollow organ that are not supported by bordering elements of the stent are reduced in size.

Furthermore, it is possible that the outer body has a larger number of cells than the inner body. The outer body can e.g. have 2×, 3× or 5× the number of cells of the inner body. The cells of the outer body can then be smaller than the cells of the inner body. The outer body can thus form a dense mesh that surrounds the inner body.

Alternatively, the outer body can also have fewer cells than the inner body. Thus, the outer body can e.g. have less than 80%, 50%, or 30% of the cells of the inner body. The cells of the outer body can then be designed larger than the cells of the inner body.

In accordance with a further embodiment, the outer body comprises, in particular at an outer side, rails that preferably extend along the longitudinal direction. The rails can enable an easier extension of the stent from an insertion set of instruments. The insertion of the stent can again be improved in this manner. The rails are preferably arranged distributed running around the longitudinal direction at the outer side of the outer body. The rails are in particular thickened so that e.g. only the rails form elevated portions at the outer side of the outer body. Since the rails are accordingly formed from the bioresorbable material or comprise the bioresorbable material, the rails are degraded in the hollow organ in the period of time and do not e.g. hinder the flexibility of the stent thereafter.

The rails can preferably be fastened to connection sections of the cells. The rails can in particular also be formed in one piece with the cells of the outer body.

Furthermore, provision can be made that the outer body comprises holding points for an insertion set of instruments. For this purpose, the outer body can, at least at one end, e.g. comprise one or more eyelets, rings or the like that enable a fastening of the insertion set of instruments.

In accordance with a further embodiment, the inner body presses against the outer body at least in the expanded state. As already mentioned above, this can in particular be the case if the inner body is a self-expanding inner body. Due to the pressure of the inner body, inner and outer bodies are each held in constant relative positions. In the compressed state, the inner body can also press against the outer body so that a fastening of the inner body to the outer body also takes place in the compressed state in this manner.

The inner body preferably presses radially outwardly against the outer body at all sides. Furthermore, the inner body can press against the outer body at least in a range of 50%, 80% or 100% of its length. In particular, the total outer side of the inner body can therefore press against the outer body.

In accordance with a further embodiment, the inner body and/or the outer body has/have fastening means to fasten the respective other body, wherein the fastening means in particular comprise hooks, projections and/or recesses. Thus, dedicated fastening means can also be provided as an alternative or in addition to the aforementioned pressure of the inner body on the outer body. The fastening means can also e.g. comprise a bonding means and/or adhesive means that fastens the inner body and the outer body to one another. The outer body preferably comprises the fastening means so that the fastening means are gradually resorbed by the body or the hollow organ. Due to said hooks or projections, the inner body can be fastened to the outer body by a hooking in or, in general, by a form fit. One of the bodies can in particular also comprise recesses into which the other body engages or in which the other body abuts in order thus to prevent a relative movement between the inner body and the outer body.

Apart from the fastening means, the outer body can be arranged completely outside the inner body. This means that the outer body does not e.g. extend into the inner body.

In accordance with a further embodiment, the inner body or the outer body is surrounded by a stent graft. Generally speaking, the inner body or the outer body can be a “covered stent”. The stent graft is in particular an artificial vessel wall that can, for example, be used in the treatment of aneurysms. However, the stent described herein can also be a “bare stent,” i.e. a stent without a stent graft.

The inner body and/or the outer body can in particular also have an active agent added to it or be coated by an active agent. Such an active agent can have an anti-proliferative effect to prevent a tissue overgrowth of the stent. For example, anti-proliferatives of the limus group, statins, P2Y12 antagonists or thrombin antagonists can be used as the active agent.

In accordance with a further embodiment, the bioresorbable material comprises zinc. The bioresorbable material preferably consists of zinc and silver or comprises zinc and silver. The bioresorbable material in particular includes 90.0 to 99.95 mass % zinc and 0.05 to 10.0 mass % silver. Such a bioresorbable material can, for example, be degraded by the body in the bloodstream within a few weeks.

Alternatively, the bioresorbable material can also comprise or consist of a polymeric material, for example, poly-lactic acid (PLA) or poly-L-lactic acid (PLLA). The bioresorbable material can also comprise a magnesium alloy or consist of a magnesium alloy. Due to the above-described use of zinc or a zinc alloy, the radiopacity of the stent can, however, be increased compared to PLA, PLLA or magnesium alloys.

The bioresorbable material can also be referred to as a biodegradable material.

A further object of the invention is a stent system comprising a stent of the type described herein and a balloon that is arranged within the inner body, wherein the balloon is configured to transfer the stent from the compressed state into the expanded state. The balloon and the stent can be connected to an insertion set of instruments. The insertion set of instruments can be part of the stent system. By means of the insertion set of instruments, the stent with the balloon located therein can be inserted in the hollow organ at its site of deployment, wherein the balloon is then expanded at the site of deployment and, during the expansion, simultaneously transfers the stent into the expanded state. The insertion set of instruments can preferably be connected to the stent at the outer body.

The statements made about the stent in accordance with the invention apply accordingly to the stent system in accordance with the invention. This in particular applies with respect to advantages and embodiments. It is furthermore understood that all the embodiments mentioned herein can be combined with one another, unless explicitly stated otherwise.

The invention will be described purely by way of example with reference to the drawings in the following. There is shown:

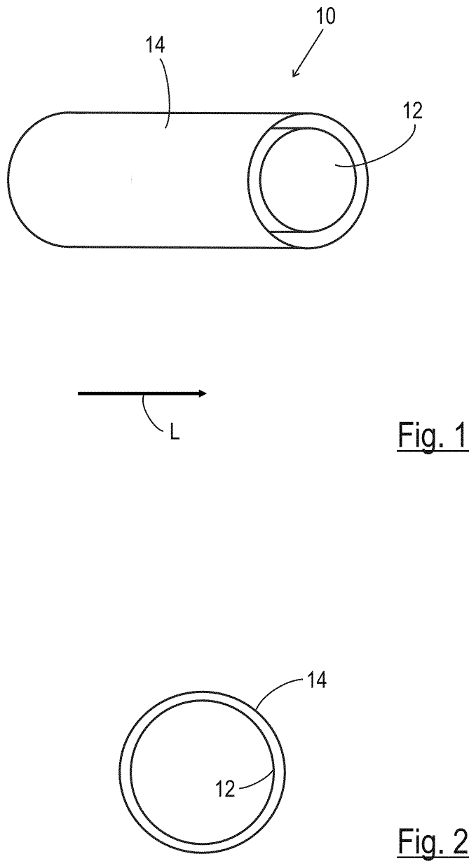

FIG. 1 schematically, a stent with an inner body and an outer body in a perspective view;

FIG. 2 schematically, the stent of FIG. 1 in a view in the direction of the longitudinal direction;

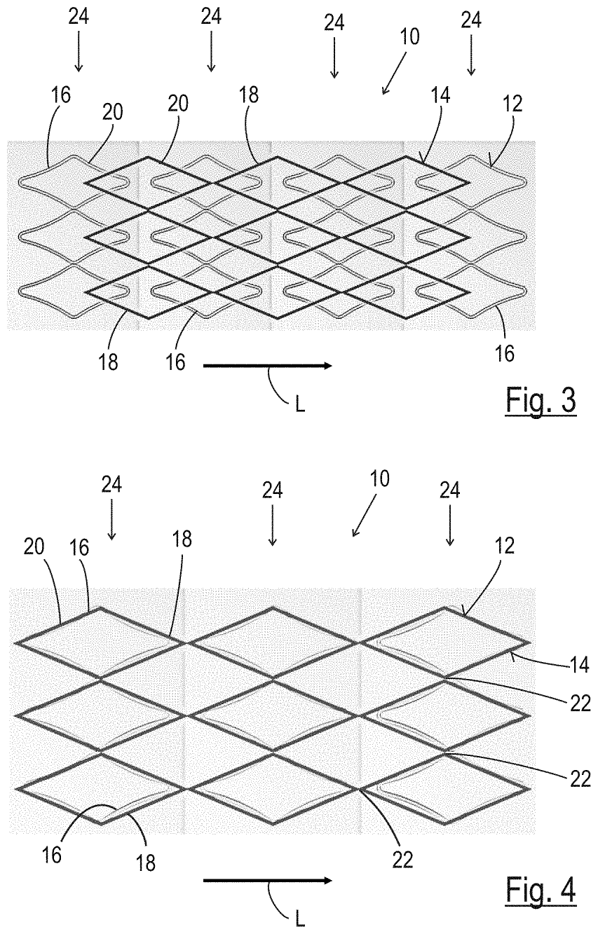

FIG. 3 cells of the inner body and the outer body of a first embodiment;

FIG. 4 cells of the inner body and the outer body of a second embodiment;

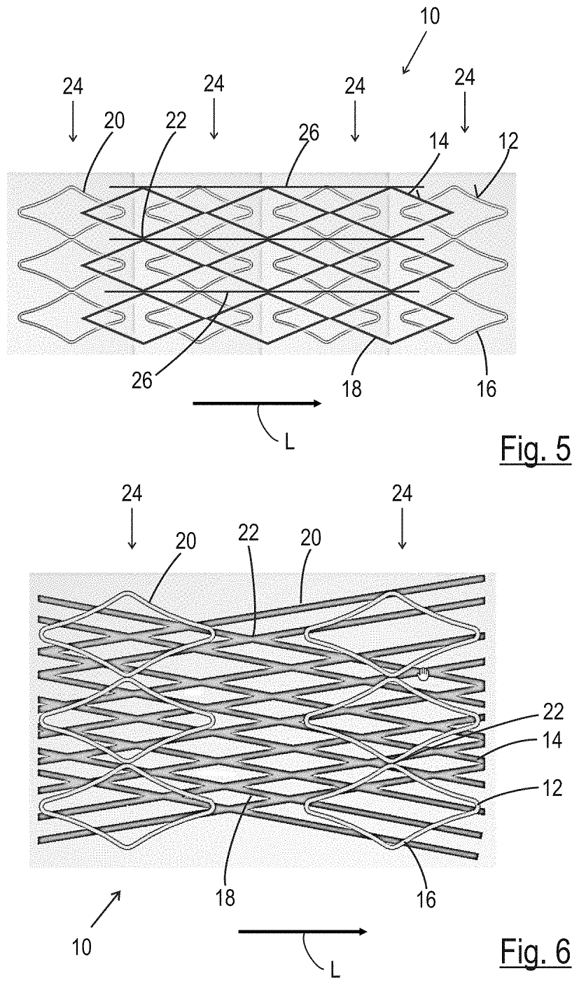

FIG. 5 an outer body with additional rails in accordance with a third embodiment; and

FIG. 6 cells of the inner body and the outer body of a fourth embodiment.

FIG. 1 shows a stent 10 in the expanded state. The stent 10 has a tubular shape and extends along a longitudinal direction L. The stent comprises a tubular inner body 12 that is surrounded by a likewise tubular outer body 14.

The inner body 12 is formed from a material that is permanently durable in the human body, for example, nitinol. In contrast, the outer body 14 is formed from a bioresorbable material, for example, from a zinc alloy.

Inner and outer bodies 12, 14 at least substantially have the same length along the longitudinal direction L. The inner body 12 has a smaller cross-sectional diameter than the outer body 14. The inner body 12 presses against the outer body 14 from the inside and is fastened to the outer body 14 by a force fit in this manner. An—alternative or additional—form-fitting fastening is not shown in the Figures.

FIG. 2 shows the stent of FIG. 1 in a view in the direction of the longitudinal direction. It can be seen that the inner and outer bodies 12, 14 form concentric rings in cross-section, i.e. that the outer body 14 surrounds the inner body 12.

FIG. 3 now shows the design of the stent 10 in more detail. The inner body 12 is formed from (inner) cells 16. Accordingly, the outer body is formed from (outer) cells 18. The cells 16, 18 each have a diamond shape and are formed from strut-like bordering elements 20.

FIG. 3 shows a section of an unwinding of the stent 10 in this respect. This means that the stent 10 can comprise even more cells 16, 18 than shown in FIG. 3. The inner cells 16 are each coupled to two other inner cells 16 via two connection sections 22. The inner cells 16 thus form separate annular sections 24 that have an equal spacing from one another in the longitudinal direction L.

The outer cells 18, in contrast, are not divided into separate annular sections. The outer cells 18 are each connected to one another via three or four connection sections 22 to form a continuous composite over the total length of the outer body 14 in this manner. Viewed in the longitudinal direction, the outer cells 18 are longer than the inner cells 16 since no spacing is provided in the longitudinal direction L between the outer cells 18.

The cells 16, 18, each shown at the top in FIG. 3, are each connected to the cells shown at the bottom in FIG. 3 to create the tubular body.

In the first embodiment of FIG. 3, the inner cells 16 and the outer cells 18 are arranged offset from one another in the longitudinal direction. This means that the connection sections 22, which connect two outer cells 18 arranged next to one another in the longitudinal direction, lie approximately centrally (e.g. above the diagonal point of intersection) above the diamond-shaped inner cell 16.

The second embodiment of the stent 10 shown in FIG. 4 differs from the first embodiment of FIG. 3 in that the inner cells 16 and the outer cells 18 are almost congruently superimposed. A small offset between the cells 16, 18 is caused by the different lengths of the cells 16, 18. In all other respects, the explanations regarding the first embodiment apply.

FIG. 5 shows a third embodiment of the stent 10. The third embodiment differs from the first embodiment only in that the outer body 14 additionally has rails 26 extending in the longitudinal direction L. The rails 26 can facilitate an insertion of the stent 10 with an insertion set of instruments. In all other respects, the explanations regarding the first embodiment apply.

Finally, FIG. 6 shows a fourth embodiment of the stent 10. In the fourth embodiment, the outer body 14 comprises significantly more cells 18 than in the first embodiment. The outer cells 18 are also usually smaller than the inner cells 16. Accordingly, the outer body 14 comprises a dense mesh of outer cells 18. In all other respects, the explanations regarding the first embodiment apply.

In all the embodiments, the outer body 14 is decomposed or resorbed in the hollow organ after a certain period of time. After that, only the inner body 12 remains in the hollow organ. Due to the separate annular sections 24, the thus remaining part of the stent 10 has a high flexibility in the longitudinal direction L, but simultaneously also has a high radial supporting effect (i.e. a high force in directions perpendicular to the longitudinal direction L).

### REFERENCE NUMERAL LIST

10 stent
12 inner body
14 outer body
16 inner cells
18 outer cells
20 bordering element
22 connection section
24 annular section
26 rail
L longitudinal direction

1. A stent for transluminal implantation into hollow organs, the stent comprising an at least substantially tubular body that extends along a longitudinal direction and that can be converted from a compressed state having a first cross-sectional diameter into an expanded state having an enlarged second cross-sectional diameter,
wherein
the tubular body comprises an inner body and an outer body, with the outer body surrounding the inner body at least regionally, with the outer body completely running around at least one section of the inner body, and
the outer body being formed from a bioresorbable material or comprises a bioresorbable material.

2. The stent in accordance with claim 1,
wherein the inner body comprises a plurality of annular sections.

3. The stent in accordance with claim 2,
wherein the annular sections are held by the outer body.

4. The stent in accordance with claim 1,
wherein at least one of the inner body and/or the outer body is an independent stent.

5. The stent in accordance with claim 1,
wherein the inner body is self-expanding and/or the outer body is balloon-expandable.

6. The stent in accordance with claim 1,
wherein the inner body comprises a plurality of cells that are defined by bordering elements formed by the inner body,
and/or
wherein the outer body comprises a plurality of cells that are defined by bordering elements formed by the outer body.

7. The stent in accordance with claim 6,
wherein cells of the inner body and/or of the outer body form a convex polygon and in particular have a diamond shape.

8. The stent in accordance with claim 6,
wherein cells of the inner body and of the outer body have the same shape or a similar shape, wherein the cells of the inner body and the outer body lie congruently on one another or are arranged offset from one another.

9. The stent in accordance with claim 1,
wherein the outer body comprises rails that extend along the longitudinal direction.

10. The stent in accordance with claim 1,
wherein the inner body presses against the outer body at least in the expanded state.

11. The stent in accordance with claim 1,
wherein at least one of the inner body and the outer body has fastening means to fasten the respective other body.

12. The stent in accordance with claim 1,
wherein one of the inner body and the outer body is surrounded by a stent graft.

13. The stent in accordance with claim 1,
wherein the bioresorbable material comprises zinc, wherein the bioresorbable material includes 90.0 to 99.95 mass % zinc and 0.05 to 10.0 mass % silver.

14. A stent system comprising a for transluminal implantation into hollow organs, the stent comprising an at least substantially tubular body that extends along a longitudinal direction and that can be converted from a compressed state having a first cross-sectional diameter into an expanded state having an enlarged second cross-sectional diameter,
wherein the tubular body comprises an inner body and an outer body, with the outer body surrounding the inner body at least regionally, with the outer body completely running around at least one section of the inner body, and
the outer body being formed from a bioresorbable material or comprises a bioresorbable material and a balloon that is arranged within the inner body and that is configured to transfer the stent from the compressed state into the expanded state.

15. The stent in accordance with claim 1,
wherein the hollow organs comprise one of blood vessels, ureters, esophagi, the colon, the duodenum, the airways and the biliary tract.

16. The stent in accordance with claim 2,
wherein the plurality of annular sections are separate from one another.

17. The stent in accordance with claim 9,
wherein the rails are arranged at an outer side of the outer body.

18. The stent in accordance with claim 11,
wherein the fastening means comprise hooks, projections, recesses and/or bonding means or adhesive means.

19. The stent in accordance with claim 13,
wherein the bioresorbable material consists of zinc and silver.
